(19) United States
(12) Patent Application Publication
(10) Pub. No.: US 2017/0362499 A1
(43) Pub. Date: Dec. 21, 2017

(54) Compositions and Methods for Preparation and Utilization of Acid-Generating Materials

(71) Applicant: Shrieve Chemical Products, Inc., The Woodlands, TX (US)

(72) Inventor: Thomas Donovan WELTON, Conroe, TX (US)

(21) Appl. No.: 15/624,330

(22) Filed: Jun. 15, 2017

Related U.S. Application Data
(60) Provisional application No. 62/351,629, filed on Jun. 17, 2016.

(51) Int. Cl. C09K 8/74; C09K 8/52; E21B 43/26; E21B 43/04; C07C 43/13; E21B 21/00; C09K 8/68; C09K 8/66; E21B 33/138; E21B 37/00; E21B 43/267; E21B 33/14

(52) U.S. Cl. CPC C09K 8/74; E21B 33/138; C09K 8/52; E21B 43/26; E21B 43/04; E21B 37/00; C07C 43/135; E21B 21/003; C09K 8/68; C09K 8/665; E21B 33/14; E21B 43/267

(57) ABSTRACT

An oilwell treatment composition comprising (i) a solubilizing agent wherein the solubilizing agent comprises a saturated compound of the formula:

H—(OCₐH₂ₐ)ₓ(OC_{b}H_{2b})_{y}—OC_{c}H_{2c+1}

where a and b are each independently 1, 3, or 4; c is 1, 2 or 3; x and y each independently, are numbers ranging from 1 to 5; (ii) a solid acid precursor and (iii) an aqueous fluid wherein the mass ratio of the solubilizing agent to the aqueous solution is within the range of about 1:3 to about 1:5 and the mass ratio of the solubilizing agent to the solid acid precursor is within the range of about 3:1 to about 2:1.

### CROSS-REFERENCE TO RELATED APPLICATIONS

The present application is a non-provisional of and claims priority to U.S. Provisional Patent Application No. 62/351,629 filed Jun. 17, 2016 and entitled “Compositions and Methods for Preparation and Utilization of Acid-Generating Materials,” which application is incorporated by reference herein in its entirety.

### TECHNICAL FIELD

The present disclosure relates to acidic materials. More particularly this disclosure relates to the preparation and utilization of acid-generating materials for use in wellbore servicing treatments.

### BACKGROUND

Acid treatment (acidizing) is one method of improving well productivity. Acidizing is commonly performed on new wells to maximize their initial productivity and on aging wells to restore productivity.

Generally, acidizing involves pumping acid into a wellbore or geologic formation that is capable of producing oil and/or gas. The purpose of any acidizing is to improve a well's productivity or injectivity. There are three general categories of acid treatments: acid washing; matrix acidizing; and fracture acidizing. In acid washing, the objective is simply tubular and wellbore cleaning without treatment of the formation. Acid washing is most commonly performed with hydrochloric acid (HCl) mixtures to clean out scale (such as calcium carbonate), rust, and other debris restricting flow in the well.

Matrix and fracture acidizing are both formation treatments. In matrix acidizing, the acid treatment is injected below the formation fracturing pressure. In fracture acidizing, acid is pumped above the formation fracturing pressure. The purpose of matrix or fracture acidizing is to restore or improve an oil or gas well's productivity by dissolving material in the productive formation that is restricting flow, or to dissolve formation rock itself to enhance existing flow paths or to create new flow paths to the wellbore.

Oftentimes acidizing has to be carried out in a controlled fashion to maximize the recovery of energy resources (i.e., hydrocarbons) while minimizing collateral damage to the formation or wellbore equipment. Thus, it would be desirable to develop improved compositions and methods for acidizing such as materials for in situ acid generation.

### SUMMARY

Disclosed herein is an oilwell treatment composition comprising (i) a solubilizing agent wherein the solubilizing agent comprises a saturated compound of the formula:

H—(OCₐH₂ₐ)ₓ(OC_{b}H_{2b})_{y}—OC_{c}H_{2c+1}

where a and b are each independently 1, 3, or 4; c is 1, 2 or 3; x and y each independently, are numbers ranging from 1 to 5; (ii) a solid acid precursor and (iii) an aqueous fluid wherein the mass ratio of the solubilizing agent to the aqueous solution is within the range of about 1:3 to about 1:5 and the mass ratio of the solubilizing agent to the solid acid precursor is within the range of about 3:1 to about 2:1.

Also disclosed herein is a method for forming a self-degrading filter cake in a subterranean formation, comprising: placing a well drill-in and servicing fluid in a subterranean formation, the well drill-in and servicing fluid comprising (i) a base fluid, (ii) a viscosifier, (iii) a fluid loss control additive, (iv) a bridging agent, and (v) an in-situ filter cake degradation composition comprising a solution formed by a solubilizing agent dissolving a solid acid precursor; and forming a filter cake upon a surface within the formation.

Also disclosed herein is an oilwell treatment composition comprising a solution formed by a solubilizing agent dissolving a solid acid precursor and a viscosifying agent.

Also disclosed herein is an oilfield treatment composition comprising a microemulsion that contains a solution made by dissolving a solid acid-precursor.

Also disclosed herein is a wellbore treatment fluid comprising i) a cyclic di-ester of an α-hydroxyacid and ii) a solubilizing agent.

### BRIEF DESCRIPTION OF THE DRAWINGS

FIG. 1 depicts infrared spectra of solutions comprising lactide and dimethyl glutarate, dimethyl succinate, and dimethyl adipate.

FIG. 2 depicts infrared spectra of solutions comprising lactide and dipropylene glycol monomethyl ether.

FIG. 3 depicts infrared spectra of solutions comprising lactide and a hydrolysable ester of formic acid and a glycol.

FIG. 4 depicts infrared spectra of solutions comprising lactide.

FIG. 5 depicts infrared spectra of solutions comprising lactide dimethyl glutarate, dimethyl succinate, and dimethyl adipate.

FIG. 6 depicts infrared spectra of solutions comprising lactide and dipropylene glycol monomethyl ether.

FIG. 7 depicts infrared spectra of solutions comprising lactide and a hydrolysable ester of formic acid and a glycol.

### DETAILED DESCRIPTION

All and any definitions and abbreviations of the terminology used in the present disclosure appear within the text where appropriate.

Disclosed herein are compositions comprising a solid acid precursor and a solubilizing agent. In an aspect, the compositions function as in situ acid generators in oilwell servicing fluids. Herein such compositions are termed Acidizing Compositions for wellbore Treatment, designated ACT.

In an aspect, the ACT comprises a solid acid precursor. A solid acid precursor suitable for use in the present disclosure is a cyclic di-ester of an α-hydroxy acid. Alpha hydroxy acids are a class of chemical compounds comprising a carboxylic acid functional group having at least one hydroxyl functional group occupying an α-position wherein the α-position is a position on the carbon atom adjacent to a carboxylic acid functional group. Nonlimiting examples of α-hydroxy acids suitable for use in the ACT include citric acid, lactic acid, methyllactic acid, glucuronic acid, glycolic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid, salts of any of the foregoing acids, or any mixtures thereof.

In an aspect, the ACT comprises the cyclic di-ester of lactic acid or lactide. In another aspect, the ACT comprises the cyclic di-ester of glycolic acid or glycolide. Referring to Table 1, the R,R; S,S; and meso-lactide structures are depicted as Structure Ia, Structure Ib, and Structure Ic, respectively while the glycolide is depicted as Structure II. In an aspect, the ACT comprises any of the structures set forth in Table 1, individually or in any combination.

TABLE 1

| | |
|---|---|
| 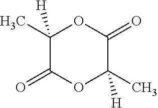 | Ia |
| 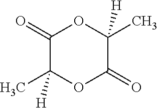 | Ib |
| 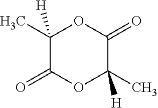 | Ic |
| 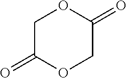 | II |

In an aspect, the solid acid precursor (e.g., lactide, glycolide) may be present in the ACT in an amount of from about 10 weight percent (wt. %) to about 90 wt. %, alternatively from about 20 wt. % to about 80 wt. % or alternatively from about 25 wt. % to about 70 wt. % based on the total weight of the composition.

In an aspect, the ACT comprises a solubilizing agent. In a non-limiting aspect, a solubilizing agent suitable for use in the present disclosure refers to a material which facilitates the formation of a stable, single-phase solution from the components of the ACT. Hence the term “stable” herein refers to the ability of the components of the ACT to form and maintain a single homogeneous phase when mixed. In a further aspect of the present disclosure, a solubilizing agent suitable for use in the present disclosure reduces or eliminates segregation of the ACT into distinct phases.

In an aspect, a solubilizing agent suitable for use in the present disclosure forms a homogenous single-phase solution after mixing the components of the ACT. In an aspect, the homogeneity of the single-phase solution may be assessed by any suitable methodology. In a further aspect the homogeneity of the single-phase solution may be assessed by visual observation of the time period required to obtain a homogeneous single phase solution after mixing the components of the ACT. In cases where the homogeneous single-phase solution later segregates into distinct phases, the homogeneity of the single-phase solution may be assessed visually in terms of the time period between when the components form a homogeneous single-phase solution and when segregation into distinct phases occurs. In an aspect of the present disclosure, an ACT may be formulated to meet the solubility needs of one or more user and/or process goals.

In a nonlimiting aspect the solubilizing agent comprises a compound of the formula:

H—(OCₐH₂ₐ)ₓ(OC_{b}H_{2b})_{y}—OC_{c}H_{2c+1}

where a and b independently, are 1, 3, or 4; c is a number ranging from 1 to 3 and x and y independently are numbers ranging from 1 to 5. In another aspect, the solubilizing agent is a saturated compound (i.e., contains no double bonds). In another aspect, the solubilizing agent comprises a mixture of compounds having Formula I wherein the compounds are constitutional isomers. In such an aspect, at least three constitutional isomers of the compounds having Formula I each comprise a single oxygen-hydrogen bond and a single alkoxy group selected from the group consisting of a methoxy group, an ethoxy group, and a propoxy group. In a further aspect, the solubilizing agent comprises dipropylene glycol monomethyl ether. In a further aspect, the solubilizing agent comprises dipropylene glycol monomethyl ether as a mixture of three constitutional isomers having the following structures:

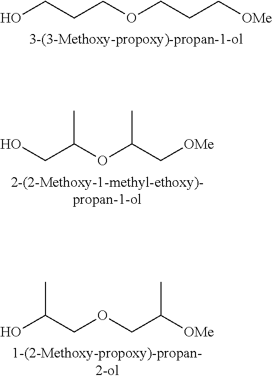

In a still further aspect, any one constitutional isomer of the solubilizing agent comprises the product of a self-condensation etherification process utilizing a single type of glycol or derivative thereof. In another aspect, the mixture of three or more constitutional isomers comprises at least two distinct glycol constitutional isomers or derivatives thereof, wherein the at least two distinct glycol constitutional isomers or derivatives thereof do not comprise ethylene glycol or a derivative thereof

In yet a further aspect the solubilizing agents may be classified as either Group A solubilizing agents or Group B solubilizing agents, and the ACT may comprise Group A solubilizing agents or Group B solubilizing agents. Herein Group A solubilizing agents comprise mutual solvents or mutual solvent precursors whereas a Group B solubilizing agent comprises a mutual solvent precursor that forms a mutual solvent and an acid. Consequently, in compositions comprising a cyclic di-ester of an α-hydroxyacid and a Group A solubilizing agent, the acid formed is derived primarily from hydrolysis of the cyclic di-ester of the α-hydroxyacid. In contrast, the compositions comprising a cyclic di-ester of an α-hydroxyacid and a Group B solubilizing agent, the acid formed is derived from both hydrolysis of the cyclic di-ester of an α-hydroxyacid and reaction of the Group B solubilizing agent (e.g., hydrolysis of the Group B solubilizing agent).

In an aspect, a Group A solubilizing agent comprises a mutual solvent. Herein, a mutual solvent is defined as a material that is soluble in more than one class of liquids such as oil, water, and acid-based treatment fluids. Given that the mutual solvent is soluble in more than one class of liquid, such materials may also be referred to as coupling agents because such materials can cause two ordinarily immiscible liquids to combine with each other. Nonlimiting examples of mutual solvents suitable for use in the present disclosure include ethylene glycol monobutylether (EGMBE) or propylene glycol monobutylether, methanol, isopropyl alcohol, alcohol ethers, aldehydes, ketones, aromatic solvents, ethylene glycol, ethylene glycol monobutyl ether, butyl carbitol, mono- or poly-hydric C₂-C₃₀ alcohols, derivatives thereof, or combinations thereof. Examples of commercially available mutual solvents include MUSOL mutual solvent sold by Halliburton Energy Services, SOL-15 sold by Fracmaster Ltd., and SUPER-SOL sold by Osca.

In an aspect, the Group A solubilizing agent is a mutual solvent precursor that does not contribute to the acid present in the composition. Herein, a mutual solvent precursor is defined as a mutual solvent or coupling agent that has been modified to provide for delayed release of the mutual solvent. Such mutual solvent precursors may also be referred to as time-delayed and/or time-released mutual solvents. Examples of modification to mutual solvents to form mutual solvent precursors include without limitation the addition of an operable functionality component or substituent, physical encapsulation or packaging, or combinations thereof. The operable functionality component or substituent may be acted upon in any fashion (e.g., chemically, physically, thermally, etc.) and under any conditions compatible with the components of the process in order to release the mutual solvent at a desired time and/or under desired conditions such as in situ wellbore conditions. In an aspect, the mutual solvent precursor may comprise at least one modified mutual solvent (e.g., having an operable functionality, encapsulation, packaging, etc.) such that when acted upon and/or in response to pre-defined conditions (e.g., in situ wellbore conditions such as temperature, pressure, chemical environment), a mutual solvent is released.

In an aspect, the Group B solubilizing agent is a liquid acid precursor such as a compound comprising a reactive ester. The reactive ester may be converted to an acidic species by hydrolysis of the ester linkage, for example, by contact with water present in situ in the wellbore. Nonlimiting examples of liquid acid precursors for use in the present disclosure include lactic acid derivatives such as methyl lactate, ethyl lactate, propyl lactate, butyl lactate; esters and/or formates that are water soluble or partially soluble such as ethylene glycol monoformate; ethylene glycol diformate; diethylene glycol diformate; glyceryl monoformate; glyceryl diformate; glyceryl triformate; triethylene glycol diformate; formate esters of pentaerythritol; esters or polyesters of glycerol including, but not limited to; tripropionin (a triester of propionic acid and glycerol); trilactin; esters of acetic acid and glycerol such as monoacetin, diacetin, and triacetin; esters of glycolic acid such as methyl glycolate, ethyl glycolate, propyl glycolate, or butyl glycolate; or esters of glycolic acid and polyols such as glycerol and glycols; aliphatic polyesters; poly(lactides); poly(glycolides); poly(ε-caprolactones); poly(hydroxybutyrates); poly(anhydrides); aliphatic polycarbonates; poly(amino acids); and polyphosphazenes; or copolymers thereof: poly(ortho esters); orthoesters (which may also be known as “poly ortho ethers” or “ortho ethers”); esters of oxalic acid; esters of propionic acid; esters of butyric acid; esters of monochloroacetic acid; esters of dichloroacetic acid; esters of trichloroacetic acid; derivatives thereof; or combinations thereof. Other suitable liquid acid precursors include halide esters and esters of nitric acid, esters of sulphuric acid, esters of sulphonic acid, esters of sulphinic acid, esters of phosphoric acid, esters of phosphorous acid, esters of phosphoric acid, esters of phosphonic acid, esters of phosphinic acid, esters of sulphamic acid, derivatives thereof, or combinations thereof.

In an aspect, the Group B solubilizing agent comprises an esterified mutual solvent. In such aspects, the ester linkage may be hydrolyzed to release the mutual solvent and an acidic species (e.g., acetic acid, formic acid). In an aspect, the Group B solubilizing agent comprises a glycol ether ester, and upon hydrolysis of the ester linkage a glycol ether mutual solvent is released. Examples of glycol ether esters suitable for use in this disclosure include without limitation butyl glycol acetate, butyl diglycol acetate, butyl triglycol acetate, butyl glycol dimethoxyacetal, isooctanol acetate, isopropanol acetate, 1-methoxy-2-propanol acetate and the corresponding acetals, propionates, and the like; or combinations thereof.

In an aspect, a Group B solubilizing agent increases the acidity of the composition. For example, the Group B solubilizing agent may provide an amount of an organic acid such as acetic acid, formic acid, lactic acid, glycolic acid, glutaric acid, succinic acid, adipic acid, or combinations thereof. For example, the Group B solubilizing agent may comprise dimethyl glutarate, dimethyl succinate, dimethyl adipate, or combinations thereof. Commercial examples of a Group B solubilizing agent include without limitation RHODIASOLVE RPDE and FLEXISOLVE DBE esters.

In an aspect, the ACTs of the present disclosure comprise solutions having a concentration of the solubilizing agent of equal to or greater than about 30 wt. %, alternatively about 40 wt. %, alternatively about 50 wt. %, or alternatively about 60 wt. % based on the total weight of the composition. In a further aspect, the ACTs comprise a saturated solution of the solubilizing agent.

In an aspect, the ACT comprises an aqueous fluid. Aqueous fluids that may be used in conjunction with the ACTs described in the present disclosure include any aqueous fluid suitable for use in subterranean applications. For example, the aqueous fluid may comprise sea water, tap water, freshwater, produced water, brine or combinations thereof. In an aspect, the brine comprises a saturated solution of an inorganic salt (e.g., sodium bromide) consisting of monovalent cations, polyvalent cations or combinations thereof and anions. In a non-limiting aspect, the anions comprise chlorides, bromides, or combinations thereof. Non-limiting examples of suitable brines include sodium chloride, sodium bromide, calcium chloride, zinc bromide, sodium formate, potassium formate, cesium formate, derivatives thereof, or combinations thereof. The specific brine used may be dictated by the desired density of the resulting ACT. Denser brines may be useful in some instances. The density of the aqueous fluid, and likewise the density of the ACT, may be selected and adjusted as recognized by one skilled in the art with the benefit of this disclosure.

In an aspect, the ACT comprises an aqueous fluid which is present in an amount that constitutes the balance of the ACT after considering the amount of the other components. In an aspect, the ACT comprises a brine which may be present in an amount of from about 40 wt. % to about 90 wt. %, alternatively from about 50 wt. % to about 80 wt. % or alternatively from about 50 wt. % to about 70 wt. % based on the total weight of the ACT.

In an aspect, an ACT of the type disclosed herein comprises a cyclic di-ester of an α-hydroxyacid (e.g., lactide, glycolide) and a solubilizing agent. Nonlimiting examples of ACTs of this disclosure include lactide and a Group A solubilizing agent; lactide and a Group B solubilizing agent; glycolide and a Group A solubilizing agent; glycolide and a Group B solubilizing agent; lactide and glycolide and a Group A solubilizing agent; lactide and glycolide and a Group B solubilizing agent; lactide and EGMBE; glycolide and EGMBE; lactide and propylene glycol monobutylether; glycolide and propylene glycol monobutylether; lactide and dipropylene glycol monomethyl ether; glycolide and dipropylene glycol monomethyl ether; lactide and methanol; glycolide and methanol; lactide and isopropyl alcohol; glycolide and isopropyl alcohol; lactide and alcohol ethers; lactide and alcohol ethers; lactide and aldehydes; glycolide and aldehydes; lactide and ketones; glycolide and ketones; lactide and aromatic solvents; glycolide and aromatic solvents; lactide and methyl lactate; glycolide and methyl lactate; lactide and ethyl lactate; glycolide and ethyl lactate; lactide and propyl lactate; glycolide and propyl lactate; lactide and butyl lactate; glycolide and butyl lactate; lactide and ethylene glycol monoformate; glycolide and ethylene glycol monoformate; lactide and ethylene glycol diformate; glycolide and ethylene glycol diformate; lactide and ethylene glycol diformate; glycolide and ethylene glycol diformate; lactide and glyceryl monoformate; glycolide and glyceryl monoformate; lactide and glyceryl diformate; glycolide and glyceryl diformate; lactide and glyceryl triformate; glycolide and glyceryl triformate; lactide and triethylene glycol diformate; glycolide and triethylene glycol diformate; lactide and tripropionin; glycolide and tripropionin; lactide and monoacetin; glycolide and monoacetin; lactide and diacetin; glycolide and diacetin; lactide and triacetin; glycolide and triacetin; lactide and esters of glycolic acid; glycolide and esters of glycolic acid; lactide and glycerol and/or glycols; glycolide and glycerol and/or glycols; lactide and aliphatic polyesters; glycolide and aliphatic polyesters; lactide and poly(lactides); glycolide and poly(lactides); lactide and poly(glycolides); glycolide and poly(glycolides); lactide and poly(ε-caprolactones); glycolide and poly(ε-caprolactones); lactide and poly(hydroxybutyrates); glycolide and poly(hydroxybutyrates); lactide and poly(anhydrides); glycolide and poly(anhydrides); lactide and aliphatic polycarbonates; glycolide and aliphatic polycarbonates; lactide and poly(amino acids); glycolide and poly(amino acids); lactide and polyphosphazenes; glycolide and polyphosphazenes; lactide and poly(ortho esters); glycolide and poly(ortho esters); lactide and orthoesters; glycolide and orthoesters; lactide and esters of oxalic acid; glycolide and esters of oxalic acid; lactide and halide esters; glycolide and halide esters; lactide and esters of nitric acid; glycolide and esters of nitric acid; lactide and esters of sulphuric acid; glycolide and esters of sulphuric acid; lactide and esters of sulphonic acid; glycolide and esters of sulphonic acid; lactide and esters of sulphinic acid; glycolide and esters of sulphinic acid; lactide and esters of phosphoric acid; glycolide and esters of phosphoric acid; lactide and esters of phosphonic acid; glycolide and esters of phosphonic acid; lactide and esters of phosphinic acid; glycolide and esters of phosphinic acid; lactide and esters of sulphamic acid; and glycolide and esters of sulphamic acid.

In an aspect, the ACT comprises a solubilizing agent, an aqueous fluid and a solid acid precursor wherein the mass ratio of the solubilizing agent to aqueous fluid is from about 1:2 to about 1:6 or alternatively from about 1:3 to about 1:5 and a mass ratio of solubilizing agent to solid acid precursor of from about 4:1 to about 1:1, alternatively of from about 3:1 to about 2:1.

In an aspect, the ACT comprises the cyclic di-ester of an α-hydroxyacid in an amount of from about 15 weight percent (wt. %) to about 85 wt. %, alternatively from about 25 wt. % to about 75 wt. %, or alternatively from about 40 wt. % to about 60 wt. % based on the total weight of the ACT and the solubilizing agent is present in an amount of from about 15 wt. % to about 85 wt. % based on based on the total weight of the ACT.

In an aspect, ACTs of the type disclosed herein form stable solution comprising a single visually homogenous phase, within about 5 minutes, alternatively within about 30 minutes, alternatively within about 1 hour, or alternatively within about 90 minutes after mixing of the solution components. In such aspects, the ACT remains stable (i.e., single phase) for at least about 24 hours, alternatively for at least about 48 hours after sitting at ambient temperature and pressure conditions.

An ACT of the type disclosed herein may function as an oilwell servicing fluid or be a component of an oilwell servicing fluid and be further used to perform an associated oilwell service or treatment. Oilwell servicing fluids are prepared and introduced into an oilwell (e.g., pumped downhold) where the oilwell servicing fluid may contact one or more mechanical components in the wellbore (e.g. wellbore casing, completion equipment, and/or production equipment), the oilwell itself or component thereof (e.g., a wellbore wall or a filter cake disposed thereon), and/or the formation surrounding the oilwell and penetrated thereby. The components of the ACT may be combined using any mixing device compatible with the composition. In an aspect, the components of the composition are combined at the well site; alternatively, the components of the composition are combined off-site and are transported to and used at the well site. In an aspect, an ACT or an oilwell servicing fluid comprising an ACT is placed into a wellbore. Once in the wellbore the ACT or oilwell servicing fluid comprising an ACT contacts water or a water-containing material. The water or water-containing material may be a naturally occurring material in the wellbore. Alternatively, water or a water-containing material is introduced to the wellbore subsequent to introduction of the ACT or oilwell servicing fluid comprising an ACT. Contacting of the ACT or oilwell servicing fluid comprising an ACT with water or a water-containing material may initiate lysis of one or more compounds present in the ACT. In some aspects, the ACT upon contact with water will affect the release of one or more acidic species from the ACT and will decrease the pH of the composition. In some aspects, a decrease in the pH accelerates hydrolysis of ester bonds present in the ACT resulting in the release of additional acidic species. The in-situ generation of acid may be a component of an oilwell servicing fluid or oilwell treatment composition, for example an acid washing treatment; a matrix acidizing treatment; a fracture acidizing treatment; a self-degrading filtercake; a drilling mud; a breaker fluid; a spacer fluid; or combinations thereof.

In an aspect, the treatment of an oilwell with an ACT or oilwell servicing fluid comprising an ACT results in the in situ generation of acid. Thus, the pH of the composition at the time of placement (e.g., pumping) down hole may not be as low (i.e., may be less acidic) than would be the case if an aqueous solution of the acidic species was pumped into the well bore. As such, acid generation of the ACT or oilwell servicing fluid comprising the ACT may be delayed for some time period to allow for the formation of a sufficient amount of acid to carry out the intended function.

In some aspects, the oilwell may be shut in for a period of time subsequent to introduction of the ACT or oilwell servicing fluid comprising the ACT to the wellbore. For example, the oilwell may be shut-in for a time of from equal to or greater than about 2 to about 3 hours, alternatively equal to or greater than about 24 hours, alternatively from equal to or greater than about 2 to about 5 days. In an aspect, an ACT or oilwell servicing fluid comprising an ACT generates acid sufficient to carry out the intended function in a period of time that allows for positioning of the material downhole at some user or process desired location, e.g. at a particular depth in the wellbore. As will be understood by the ordinarily skilled artisan, the time for generation of a sufficient acid concentration will depend to some extent on the wellbore temperature which may range from about 100° F. to about 500° F., alternatively from about 100° F. to about 400° F., or alternatively from about 125° F. to about 400° F.

In an aspect, the ACT may be a component of a breaker fluid. Breaker fluids herein refer to oilwell servicing fluids used to destroy the integrity of a residual filter cake created during the drilling process by removing some or all drilling fluid components that form the filter cake. As such, the ACT may function to facilitate the removal of the filter cake and thus aid in the return of permeability to the wellbore. Filter cake breaking may be classified into two general approaches: “dispersion” of the filter cake or “dissolution” of the filter cake. In the case of dispersion, the primary filter cake components (fluid loss agents) are destroyed, typically leaving the bridging agents (often calcium carbonate) to flow back out of the wellbore or screen-out or become incorporated into a gravel pack, whereas in the case of dissolution, both the fluid loss agents and the bridging agents are desirably destroyed.

Dissolution comprises breaking of bridging agents in addition to fluid loss agents and conventionally relies on acid dissolution of the bridging agents coupled with oxidative or enzymatic breaking of the fluid loss agents. Consequently, a breaker fluid may comprise the ACT, a polymer degrading enzyme, chelants, an oxidizing agent, and a surfactant.

In some aspects, the chelating agent in a breaker fluid comprising the ACT can be a polydentate chelator such that multiple bonds are formed with the complexed ion, e.g., calcium from the calcium carbonate. Polydentate chelators suitable for use in the breaker fluids of the present disclosure may include, for example, salts of ethylenediaminetetraacetic acid (EDTA), diethylenetriaminepentaacetic acid (DTPA), nitrilotriacetic acid (NTA), ethylene glycol-bis(2-aminoethyl)-N,N,N′,N′-tetraacetic acid (EGTA), 1,2-bis(o-aminophenoxy)ethane-N,N,N′,N′-tetraaceticacid (BAPTA), cyclohexanediaminete-traacetic acid (CDTA), triethylenetetraaminehexaacetic acid (TTNA), N-(2-Hydroxyethyl)ethylenediamine-N,N′,N′-triacetic acid (HEDTA), glutamic-N,N-diacetic acid (GLDA), ethylene-diamine tetra-methylene sulfonic acid (EDTMS), diethylene-triamine penta-methylene sulfonic acid (DETPMS), amino tri-methylene sulfonic acid (ATMS), ethylene-diamine tetra-methylene phosphonic acid (EDTMP), diethylene-triamine penta-methylene phosphonic acid (DETPMP), amino tri-methylene phosphonic acid (ATMP), or mixtures thereof.

In addition to a chelating agent, the breaker fluids of the present disclosure may include at least one polymeric degradation agent for breaking of polymeric fluid loss agents within the filter cake. Such polymeric degradation agents may include enzymes and/or oxidants. Enzymes suitable for use in the present disclosure include the hemicellulases. Alternatively, an oxidant may be included in the breaker fluid, to aid in breaking or degradation of polymeric additives present in a filter cake. Examples of such oxidants may include any one of those oxidative breakers known in the art to disrupt filter cakes. Such compounds may include peroxides (including peroxide adducts), other compounds including a peroxy bond such as persulphates, perborates, percarbonates, perphosphates, and persilicates, and other oxidizers such as hypochlorites, which may optionally be encapsulated as taught by U.S. Pat. No. 6,861,394; the relevant portions of which are incorporated by reference herein.

In some aspects, the breaker fluid comprising the ACT can also include a brine. Such brines include, zinc bromide, sodium bromide, calcium bromide, sodium or potassium chloride, ammonium chloride, calcium chloride, seawater, cesium formate, sodium formate, potassium formate, and combinations thereof.

In an aspect, the ACT is a component of a drilling-mud and may facilitate in the formation of a self-degrading filtercake. The drilling mud comprises one or more liquid components in an amount sufficient to form a pumpable slurry, and the one or more liquid components may be aqueous liquids (e.g., aqueous muds), non-aqueous liquids (e.g., oil based muds), or combinations thereof (emulsion muds). The drilling mud may additionally comprise components such as weighting materials that may be used to increase the density of the mud in order to equilibrate the pressure between the wellbore and formation when drilling through particularly pressurized zones. The drilling mud may also comprise corrosion inhibitors such as iron oxide, aluminum bisulfate, zinc carbonate, and zinc chromate which function to protect pipes and other metallic components from acidic compounds encountered in the formation. The drilling mud may also comprise dispersants, including iron lignosulfonates, which function to break up solid clusters into small particles so they can be carried by the fluid. The drilling mud may also comprise flocculants, such as acrylic polymers, which function to cause suspended particles to group together so they can be removed from the fluid at the surface. The drilling mud may also comprise surfactants, like fatty acids and soaps, which function to defoam and emulsify the mud. The drilling mud may also comprise biocides, typically organic amines, chlorophenols, or formaldehydes, which function to kill bacteria and help reduce the souring of drilling mud. The drilling mud may also comprise fluid loss additives such as starch and organic polymers which function to limit the loss of drilling mud to under-pressurized or high-permeability formations.

Drilling muds may additionally comprise conventional additives such as bridging agents, viscosifiers, and the like. Herein a “bridging agent” refers to solids added to a drilling fluid to bridge across the pore throat or fractures of an exposed rock thereby building a filter cake to prevent loss of whole mud or excessive filtrate. Herein, a “viscosifier” refers to a material used to increase the viscosity of the oilwell servicing fluids. Nonlimiting examples of viscosifying agents include guar gums, high-molecular weight polysaccharides composed of mannose and galactose sugars, or guar derivatives such as hydroxypropyl guar (HPG), carboxymethyl guar (CMG), or carboxymethylhydroxypropyl guar (CMHPG). Cellulose derivatives may be used such as hydroxyethylcellulose (HEC) or hydroxypropylcellulose (HPC) or carboxymethylhydroxyethylcellulose (CMHEC). Any useful polymer may be used in either crosslinked form, or without crosslinker in linear form. Xanthan, diutan, and scleroglucan, three biopolymers, have been shown to be useful as viscosifying agents. Surfactants that may be included in the drilling muds are cationic surfactants, anionic surfactants, zwitterionic surfactants, amphoteric surfactants, nonionic surfactants, and combinations thereof.

In additional or alternative aspects, the ACT may be a component of an oilwell servicing fluid formulated as a gravel pack fluid. In such aspects the gravel pack fluid may be employed in a gravel packing treatment. Gravel packing treatments are used, inter alia, to reduce the migration of unconsolidated formation particulates into the wellbore. In gravel packing operations, particulates, referred to as gravel, are carried to a wellbore in a subterranean producing zone by a servicing fluid known as carrier fluid. That is, the particulates are suspended in a carrier fluid, which may be viscosified, and the carrier fluid is pumped into a wellbore in which the gravel pack is to be placed. As the particulates are placed in the zone, the carrier fluid leaks off into the subterranean zone and/or is returned to the surface. The resultant gravel pack acts as a filter to separate formation solids from produced fluids while permitting the produced fluids to flow into and through the wellbore. The ACT may facilitate the separation of formation solids from the produced fluids. When installing the gravel pack, the gravel is carried to the formation in the form of a slurry by mixing the gravel with a viscosified carrier fluid. Such gravel packs may be used to stabilize a formation while causing minimal impairment to well productivity. The gravel, inter alia, acts to prevent the particulates from occluding the screen or migrating with the produced fluids, and the screen, inter alia, acts to prevent the gravel from entering the wellbore.

In an aspect, the ACT is used as or is a component of a spacer fluid. Spacer fluids are liquids used to physically separate one special-purpose liquid (e.g., oilwell servicing fluid) from another. In an aspect, the ACT facilitates the separation of fluids, wellbore cleaning, and/or the formation of stable microemulsions.

Special-purpose liquids are typically subject to contamination, so a spacer fluid compatible with each is used between the two. Spacers are used primarily when changing mud types or changing from mud to a completion fluid. In the former, an oil-based fluid must be kept separate from a water-based fluid. Another common use of spacers is to separate mud from cement during cementing operations. For cementing, a chemically treated aqueous spacer or sequence of spacers usually separates drilling mud from the cement slurry subsequently pumped downhole. In an aspect, the ACT facilitates the separation of fluids. Cleaning spacers are also extensively used to clean the casing, riser and other equipment after drilling a section of wellbore. Cleaning spacers not only remove the remaining drilling fluid from the wellbore, but also cuttings. Methods and compositions herein have the advantages of reduced potential damage to the well, by avoiding plugging of completion equipment (stand alone screens, expandable screens, gravel packs, etc.) by residual debris, and consequently increased hydrocarbon recovery, and/or increased water injection rate, as compared with an otherwise identical method and composition absent the spacer fluid. In some aspects, the spacer fluid is a microemulsion. Microemulsions are thermodynamically stable, macroscopically homogeneous mixtures of at least three components: a nonpolar phase and a polar phase (usually, but not limited to, water and organic phase) and at least one surfactant, often more than one surfactant, for instance with a cosurfactant such as an alcohol, glycol or phenol, or their alkoxy derivatives, particularly when ionic surfactants are used

Surfactants suitable for creating the spacer fluids (e.g. single phase microemulsions) using these methods herein include, but are not necessarily limited to non-ionic, anionic, cationic and amphoteric surfactants and in particular, blends thereof. Co-solvents or co-surfactants such as alcohols are optional additives used in the microemulsion formulation. Suitable nonionic surfactants include, but are not necessarily limited to, alkyl polyglycosides, sorbitan esters, methyl glucoside esters, alcohol ethoxylates, or polyglycol esters. In one non-restrictive version, polyglycol esters are particularly suitable, and in another non-limiting embodiment there is an absence of alkyl polyglycosides. Suitable cationic surfactants include, but are not necessarily limited to, arginine methyl esters, alkanolamines and alkylenediamides. Suitable anionic surfactants include, but are not necessarily limited to, alkali metal alkyl sulfates, alkyl or alkylaryl sulfonates, linear or branched alkyl ether sulfates and sulfonates, alcohol polypropoxylated and/or polyethoxylated sulfates, alkyl or alkylaryl disulfonates, alkyl disulfates, alkyl sulphosuccinates, alkyl ether sulfates, linear and branched ether sulfates, and mixtures thereof. In one non-limiting embodiment at least two surfactants in a blend may be used to create single phase microemulsions, as well as the other spacer fluids. Suitable surfactants may also include surfactants containing a non-ionic spacer-arm central extension and an ionic or nonionic polar group. The non-ionic spacer-arm central extension may be the result of polypropoxylation, polyethoxylation, or a mixture of the two, in non-limiting embodiments.

In an aspect, the ACT may be a component of an oilwell servicing fluid formulated for use in offshore drilling. An ACT may afford the formation of an acidic species under the conditions of lower temperature formations frequently encountered in offshore drilling. Offshore drilling is a mechanical process where a wellbore is drilled below the seabed. It is typically carried out in order to explore for and subsequently extract petroleum which lies in rock formations beneath the seabed. Most commonly, the term is used to describe drilling activities on the continental shelf, though the term can also be applied to drilling in lakes, inshore waters and inland seas.

In an aspect, the ACT is used as or is a component of a fracturing fluid. Hydraulic fracturing is a common stimulation technique used to enhance production of oil and gas from hydrocarbon containing reservoirs. In a typical hydraulic fracturing operation, fracturing fluid is pumped at high pressures and high rates through a wellbore penetrating a subterranean formation to initiate and propagate hydraulic fractures in the formation. Subsequent steps typically include adding particulate matter known as proppant to the fracturing fluid (e.g., graded sand, ceramic particles, bauxite, or resin coated sand) which is carried by the fracturing fluid into the fractures. The proppant deposits into the fractures, forming a permeable “proppant pack”. Once the fracture treatment is completed, the fracture closes onto the proppant pack allowing for maintenance of the fracture, thereby providing a pathway for hydrocarbons in the formation to flow more easily into the wellbore for recovery.

The foregoing has outlined rather broadly the features and technical advantages of the present subject matter in order that the detailed description of the subject matter that follows may be better understood. Additional features and advantages of the subject matter will be described hereinafter that form the subject of the claims of the subject matter. It should be appreciated by those skilled in the art that the conception and the specific aspects disclosed may be readily utilized as a basis for modifying or designing other structures for carrying out the same purposes of the present subject matter. It should also be realized by those skilled in the art that such equivalent constructions do not depart from the spirit and scope of the subject matter as set forth in the appended claims. It should also be realized by those skilled in the art that the selection of an ACT for use in a particular treatment fluid can be tailored based upon a variety of parameters including but not limited to the desired solubility of the ACT in the final fluid, the treatment conditions and other operational considerations.

While these aspects may be disclosed under these headings, the heading does not limit the disclosure found therein. Additionally, the various aspects and embodiments disclosed herein can be combined in any manner.

It is to be understood that herein the values provided are for the amount (e.g., wt. %) of a component or reactant used in the preparation of compositions of the type disclosed herein (e.g., lactide). The final material after all processing steps (e.g., lactide) may not have the individual components (e.g., lactide) discernable chemically or physically from the other components utilized in the preparation of the final composition.

The subject matter of the present disclosure having been generally described, the following examples are given as particular aspects of the disclosure and to demonstrate the practice and advantages thereof. It is understood that the examples are given by way of illustration and are not intended to limit the specification or the claims to follow in any manner.

The terms “a,” “an,” and “the” are intended, unless specifically indicated otherwise, to include plural alternatives, e.g., at least one. Herein, while compositions and processes are described in terms of “comprising” various components or steps, the compositions and methods can also “consist essentially of” or “consist of” the various components or steps. A particular feature of the disclosed subject matter can be disclosed as follows: Feature X can be A, B, or C. It is also contemplated that for each feature the statement can also be phrased as a listing of alternatives such that the statement “Feature X is A, alternatively B, or alternatively C” is also an aspect of the present disclosure whether or not the statement is explicitly recited.

While various aspects of the present disclosures have been shown and described, modifications thereof can be made by one skilled in the art without departing from the spirit and teachings of the disclosure. The aspects of the present disclosures described herein are exemplary only, and are not intended to be limiting. Many variations and modifications of the disclosure disclosed herein are possible and are within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure of the present disclosure. Thus, the claims are a further description and are an addition to the aspect of the present disclosures of the present disclosure. The discussion of a reference in the present disclosure is not an admission that it is prior art to the present disclosure, especially any reference that may have a publication date after the priority date of this application. The present disclosures of all patents, patent applications, and publications cited herein are hereby incorporated by reference, to the extent that they provide exemplary, procedural or other details supplementary to those set forth herein.

All publications, patent applications, and patents mentioned herein are incorporated by reference in their entirety. In the event of conflict, the present specification, including definitions, is intended to control. With respect to all ranges disclosed herein, such ranges are intended to include any combination of the mentioned upper and lower limits even if the particular combination is not specifically listed.

The following enumerated aspects are provided as non-limiting examples.

A first aspect which is an oilwell treatment composition comprising (i) a solubilizing agent wherein the solubilizing agent comprises a saturated compound of the formula:

H—(OCₐH₂ₐ)ₓ(OC_{b}H_{2b})_{y}—OC_{c}H_{2c+1}

where a and b are each independently 1, 3, or 4; c is 1, 2 or 3; x and y each independently, are numbers ranging from 1 to 5; (ii) a solid acid precursor and (iii) an aqueous fluid wherein the mass ratio of the solubilizing agent to the aqueous solution is within the range of about 1:3 to about 1:5 and the mass ratio of the solubilizing agent to the solid acid precursor is within the range of about 3:1 to about 2:1.

A second aspect which is the composition of the first aspect wherein the solubilizing agent comprises a mixture of three or more constitutional isomers wherein each constitutional isomer comprises a single oxygen-hydrogen bond and a single alkoxy group and wherein the single alkoxy group is selected from the group consisting of methoxy, ethoxy and propoxy.

A third aspect which the composition of any of the first through second aspects wherein any one constitutional isomer comprises the product of a self-condensation etherification process utilizing a single type of a glycol or derivative thereof; wherein the mixture of the three or more constitutional isomers comprises at least two distinct glycol constitutional isomers or derivatives thereof; and wherein the at least two distinct glycol constitutional isomers or derivatives thereof do not comprise ethylene glycol or a derivative thereof.

A fourth aspect which is the composition of any of the first through third aspects wherein the solubilizing agent comprises dipropylene glycol monomethyl ether as a mixture of three constitutional isomers with the following structures:

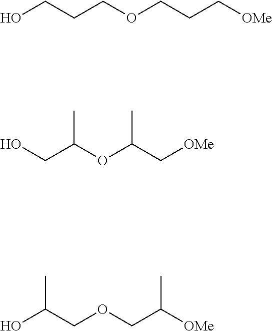

A fifth aspect which is the composition of any of the first through fourth aspect wherein the aqueous fluid comprises sea water, tap water, freshwater, produced water, brine or combinations thereof.

A sixth aspect which is the composition of the fifth aspect wherein the brine comprises a saturated solution of an inorganic salt consisting of monovalent cations, polyvalent cations or combinations thereof and anions consisting of chlorides, bromides, or combinations thereof.

A seventh aspect which is the composition of the sixth aspect wherein the inorganic salt comprises sodium bromide.

An eighth aspect which is the composition of any of the first through seventh aspects wherein the solid acid precursor is selected from the group consisting of lactide, polylactic acid, and mixtures thereof.

A ninth aspect which is the composition of any of the first through eighth aspects characterized by the formation of a single visually homogenous phase within about 5 minutes of mixing.

A tenth aspect which is the composition of the ninth aspect wherein the single homogeneous phase remains for at least 24 hours.

An eleventh aspect which is an oilwell treatment composition comprising (i) the composition of any of first through tenth aspects and (ii) dimethyl glutarate, dimethyl succinate, dimethyl adipate or combinations thereof.

A twelfth aspect which is an oilwell treatment composition comprising (i) the composition of any of first through tenth aspects and (ii) a surfactant.

A thirteenth aspect which is a breaker fluid comprising the composition of any of the first through tenth aspects, a polymer-degrading enzyme, an oxidizing agent, a surfactant and a chelant.

A fourteenth aspect which is a method of breaking a filtercake in a wellbore comprising: introducing a breaker fluid having the composition of the thirteenth aspect to the wellbore; and introducing an aqueous fluid to the wellbore.

A fifteenth aspect which is a method for forming a self-degrading filter cake in a subterranean formation, comprising placing a well drill-in and servicing fluid in a subterranean formation, the well drill-in and servicing fluid comprising (i) a base fluid, (ii) a viscosifier, (iii) a fluid loss control additive, (iv) a bridging agent, and (v) an in-situ filter cake degradation composition comprising a solution formed by a solubilizing agent dissolving a solid acid precursor; and forming a filter cake upon a surface within the formation.

A sixteenth aspect which is the method of the fifteenth aspect wherein forming a filter cake upon a surface within the formation comprises forming the filter cake upon the face of the formation itself, upon a sand screen, or upon a gravel pack.

A seventeenth aspect which is the method of any of the fifteenth through sixteenth aspects wherein the base fluid is present in the well drill-in and servicing fluid in an amount in the range of from about 20% to about 99.99% by volume.

An eighteenth aspect which is the method of any of fifteenth through seventeenth aspects wherein the viscosifier is selected from the group consisting of a biopolymer, cellulose, a cellulose derivative, guar, a guar derivative, and combinations thereof.

A nineteenth aspect which is the method of any of the fifteenth through eighteenth aspects wherein the viscosifier is present in the well drill-in and servicing fluid in an amount in the range of from about 0.2% to about 0.6% by total weight of the well drill-in and servicing fluid.

A twentieth aspect which is the method of any of the fifteenth through nineteenth aspects wherein the fluid loss control additive is present in the well drill-in and servicing fluid in an amount in the range of from about 0.01% to about 3% by total weight of the well drill-in and servicing fluid.

A twenty-first aspect which is the method of any of the fifteenth through twentieth aspects wherein the bridging agent comprises at least one of the materials selected from the group consisting of calcium carbonate, a magnesium compound, a chemically bonded ceramic bridging agent and a combination thereof.

A twenty-second aspect which is the method of any of the fifteenth through twenty-first aspects wherein the bridging agent is present in the well drill-in and servicing fluid in an amount in the range of from about 0.1% to about 32% by total weight of the well drill-in and servicing fluid.

A twenty-third aspect which is the method of any of the fifteenth through twenty-second aspects wherein the in-situ filter cake degradation composition is present in the well drill-in and servicing fluid in an amount in the range of from about 10% to about 25% by total weight of the well drill-in and servicing fluid.

A twenty-fourth aspect which is the method of the twenty-third aspect wherein the in-situ filter cake degradation composition comprises the treatment composition any of the first through the twenty-third aspects.

A twenty-fifth aspect which is an oilwell treatment composition comprising a solution formed by a solubilizing agent dissolving a solid acid precursor and a viscosifying agent.

A twenty-sixth aspect which is a method comprising introducing the treatment composition of the twenty-fifth aspect into a subterranean formation at a pressure sufficient to create a fracture in the subterranean formation.

A twenty-seventh aspect which is the composition of any of the first through tenth and twenty-fifth aspects wherein the fluid contains proppant.

A twenty-eighth aspect which is an oilwell treatment composition comprising i) a cyclic di-ester of an α-hydroxyacid and ii) a solubilizing agent.

A twenty-ninth aspect which is the composition of the twenty-eighth aspect wherein the cyclic di-ester of an α-hydroxyacid comprises R,R-lactide, S,S-lactide, meso-lactide, glycolide, or combinations thereof.

A thirtieth aspect which is a treatment composition comprising a solution made by dissolving a solid acid-precursor.

A thirty-first aspect which is the treatment composition of the thirtieth aspect wherein the solid acid-precursor is selected from the group consisting of lactide, polylactic acid, and mixtures thereof.

A thirty-second aspect which the composition of any of the thirtieth through thirty-first aspects wherein the solvent used to dissolve the solid acid-precursor also generates an acid.

A thirty-third aspect which is the composition of the thirty-second aspect wherein the solvent used to dissolve the solid acid-precursor also is a mutual solvent, mutual solvent precursor, or mixtures thereof.

A thirty-fourth aspect which is the composition of any of the thirty-second through thirty-third aspects wherein the solvent used to dissolve the solid acid-precursor also is selected from the group consisting of formic, acetic, lactic acid ester of a mutual solvent, and combinations thereof.

A thirty-fifth aspect which is the composition of any of the thirty-second through thirty-fourth aspects wherein the solvent used to dissolve the solid acid-precursor is selected from the group consisting of DPM, ethylene glycol, ethylene glycol monobutyl ether, and butyl carbitol.

A thirty-sixth aspect which is the composition of any of the thirtieth through thirty-fifth aspects in a breaker fluid further comprising a natural polymer degrading enzyme, an oxidizing agent, a surfactant, a chelant, and combinations thereof.

A thirty-seventh aspect which is the composition of any of the thirty-second through thirty-sixth aspects wherein the solvent used to dissolve the solid acid-precursor is selected from the group consisting of dimethyl glutarate, dimethyl succinate, dimethyl adipate, and combinations thereof.

A thirty-eighth aspect which is an oilfield treatment composition comprising the composition of any of the thirtieth through thirty-seventh aspects and a fluid containing dimethyl glutarate, dimethyl succinate, dimethyl adipate, or combinations thereof.

A thirty-ninth aspect which is an oilfield treatment composition comprising a microemulsion that contains a solution made by dissolving a solid acid-precursor.

A fortieth aspect which is a method comprising emplacing the breaker fluid of the thirty-sixth aspect into a wellbore drilled with a drilling fluid, the breaker fluid comprising a solution made by dissolving a solid acid-precursor.

A forty-first aspect which is the method of the fortieth aspect further comprising shutting in the well for a period of time.

A forty-second aspect which is a method of breaking a filtercake in a wellbore comprising: circulating the pre-mixed breaker fluid of the thirty-sixth aspect to the wellbore, the pre-mixed breaker fluid comprising a solution made by dissolving a solid acid-precursor; and circulating an aqueous fluid to the wellbore.

A forty-third aspect which is a method for forming a self-degrading filter cake in a subterranean formation, comprising: placing a well drill-in and servicing fluid in a subterranean formation, the well drill-in and servicing fluid comprising (i) a base fluid, (ii) a viscosifier, (iii) a fluid loss control additive, (iv) a bridging agent, and (v) an in-situ filter cake degradation composition comprising a solution made by dissolving a solid acid-precursor; and forming a filter cake upon a surface within the formation.

A forty-fourth aspect which is the method of the forty-third aspect wherein forming a filter cake upon a surface within the formation comprises forming the filter cake upon the face of the formation itself, upon a sand screen, or upon a gravel pack.

A forty-fifth aspect which is the method of any of the forty-third through forty-fourth aspects wherein the base fluid is present in the well drill-in and servicing fluid in an amount in the range of from about 20% to about 99.99% by volume.

A forty-sixth aspect which is the method of any of the forty-third through forty-fifth aspects wherein the viscosifier is selected from the group consisting of: a biopolymer, cellulose, a cellulose derivative, guar, and any guar derivative.

A forty-seventh aspect which is the method of any of the forty-third through forty-sixth aspects wherein the viscosifier is present in the well drill-in and servicing fluid in an amount sufficient to provide a desired degree of solids suspension.

A forty-eighth aspect which is the method of any of the forty-third through forty-seventh aspects wherein the viscosifier is present in the well drill-in and servicing fluid in an amount in the range of from about 0.2% to about 0.6% by total weight of the well drill-in and servicing fluid.

A forty-ninth aspect which is the method of any the forty-third through forty-eighth aspects wherein the fluid loss control additive is present in the well drill-in and servicing fluid in an amount sufficient to provide a desired degree of fluid loss control.

A fiftieth aspect which is the method of any the forty-fourth through forty-ninth aspects wherein the fluid loss control additive is present in the well drill-in and servicing fluid in an amount in the range of from about 0.01% to about 3% by total weight of the well drill-in and servicing fluid.

A fifty-first aspect which is the method of any of the forty-fourth through fiftieth aspect wherein the bridging agent comprises at least one of the following: calcium carbonate, a magnesium compound, a chemically bonded ceramic bridging agent, or a derivative thereof.

A fifty-second aspect which is the method of any forty-fourth through fifty-first aspects wherein the bridging agent is present in the well drill-in and servicing fluid in an amount sufficient to create an efficient filter cake.

A fifty-third aspect which is the method of any forty-fourth through fifty-second aspects wherein the bridging agent is present in the well drill-in and servicing fluid in an amount in the range of from about 0.1% to about 32% by total weight of the well drill-in and servicing fluid.

A fifty-fourth aspect which is the method of any of the forty-fourth through fifty-third aspects wherein the in-situ filter cake degradation composition is present in the well-drill in and servicing fluid in an amount sufficient to remove, to a desired degree, a filter cake that has been established in a subterranean formation by the well drill-in and servicing fluid.

A fifty-fifth aspect which is the method of any of the forty-fourth through fifty-fourth aspects wherein the in-situ filter cake degradation composition is present in the well drill-in and servicing fluid in an amount in the range of from about 10% to about 25% by total weight of the well drill-in and servicing fluid.

A fifty-sixth aspect which is an oilfield treatment composition comprising a solution made by dissolving a solid acid-precursor and a viscosifying agent.

A fifty-seventh aspect which is the composition of the fifty-sixth aspect wherein the fluid is introduced into a subterranean formation at a pressure sufficient to create a fracture in the subterranean formation.

A fifty-eighth aspect which is the composition of any of the fifty-sixth through fifty-seventh aspects wherein the fluid contains proppant.

A fifty-ninth aspect which is a wellbore treatment fluid comprising i) a cyclic di-ester of an α-hydroxyacid and ii) a solubilizing agent.

A sixtieth aspect which is the fluid of the fifty-ninth aspect wherein the cyclic di-ester of an α-hydroxyacid comprises R,R-lactide, S,S-lactide, meso-lactide, glycolide, or combinations thereof.

A sixty-first aspect which is the fluid of any of the fifty-ninth through sixtieth aspects wherein the solubilizing agent comprises a Group A solubilizing agent, a Group B solubilizing agent, or combinations thereof.

A sixty-second aspect which is the fluid of the sixty-first aspect wherein the Group A solubilizing agent comprises ethylene glycol monobutylether (EGMBE); propylene glycol monobutylether; methanol; isopropyl alcohol; alcohol ethers; aldehydes; ketones; aromatic solvents; dipropylene glycol methyl ether; ethylene glycol; ethylene glycol monobutyl ether; butyl carbitol; mono-or poly-hydric C₂-C₃₀ alcohols; derivatives thereof; or combinations thereof.

A sixty-third aspect which is the fluid of the sixty-first aspect wherein the Group B solubilizing agent comprises methyl lactate, ethyl lactate; propyl lactate; butyl lactate; esters and/or formates that are water soluble or partially soluble; ethylene glycol monoformate; ethylene glycol diformate; diethylene glycol diformate; glyceryl monoformate; glyceryl diformate; glyceryl triformate; triethylene glycol diformate; formate esters of pentaerythritol; esters or polyesters of glycerol; tripropionin; trilactin; esters of acetic acid and glycerol; monoacetin, diacetin; triacetin; esters of glycolic acid; methyl glycolate; ethyl glycolate; propyl glycolate; butyl glycolate; esters of glycolic acid and polyols; glycerol and glycols; aliphatic polyesters; poly(lactides); poly(glycolides); poly(ε-caprolactones); poly(hydroxybutyrates); poly(anhydrides); aliphatic polycarbonates; poly(amino acids); polyphosphazenes; poly(ortho esters); orthoesters (which may also be known as “poly ortho ethers” or “ortho ethers”); esters of oxalic acid; esters of propionic acid; esters of butyric acid; esters of monochloroacetic acid; esters of dichloroacetic acid; esters of trichloroacetic acid; halide esters, esters of nitric acid; esters of sulphuric acid; esters of sulphonic acid; esters of sulphinic acid; esters of phosphoric acid; esters of phosphorous acid; esters of phosphoric acid; esters of phosphonic acid; esters of phosphinic acid; esters of sulphamic acid; dimethyl glutarate; dimethyl succinate; dimethyl adipate; or combinations thereof.

A sixty-sixth aspect which is a method of servicing a wellbore comprising placing the treatment fluid of the fifty-ninth aspects into the wellbore.

### EXAMPLES

The subject matter of the present disclosure having been generally described, the following examples are given as particular aspects of the disclosure and to demonstrate the practice and advantages thereof. It is understood that the examples are given by way of illustration and are not intended to limit the specification or the claims to follow in any manner.

### Example 1

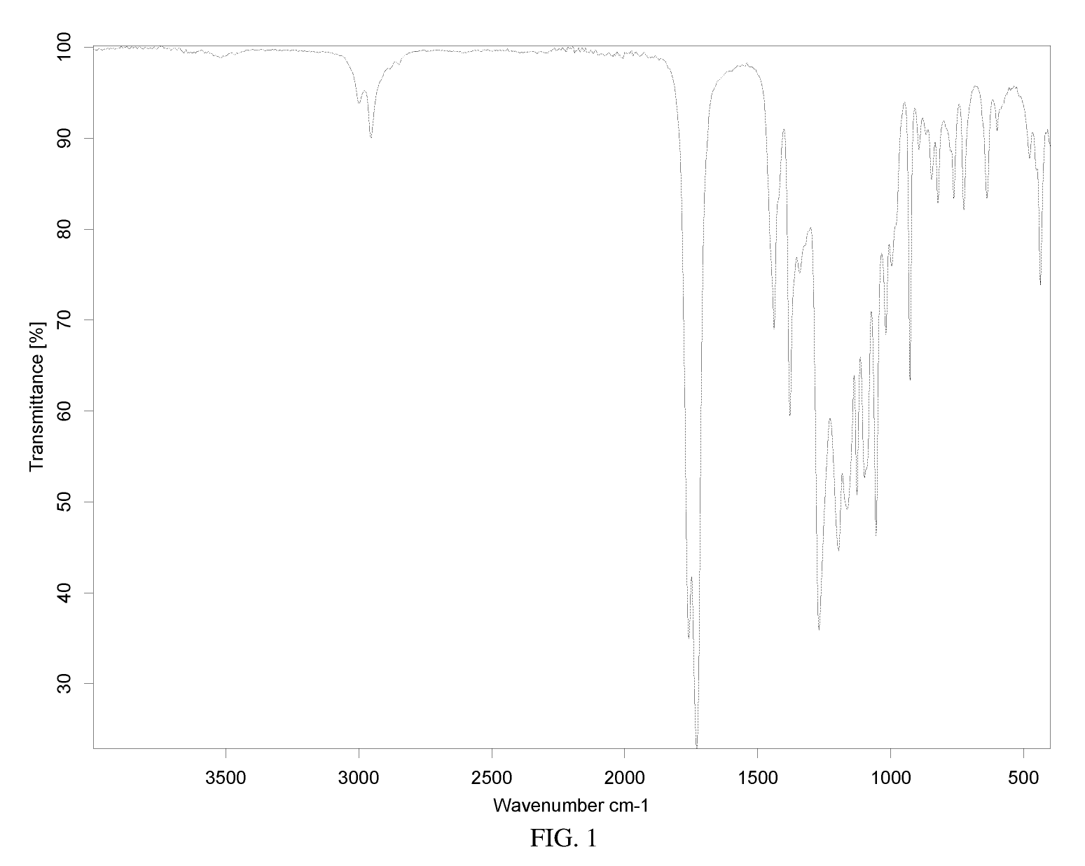

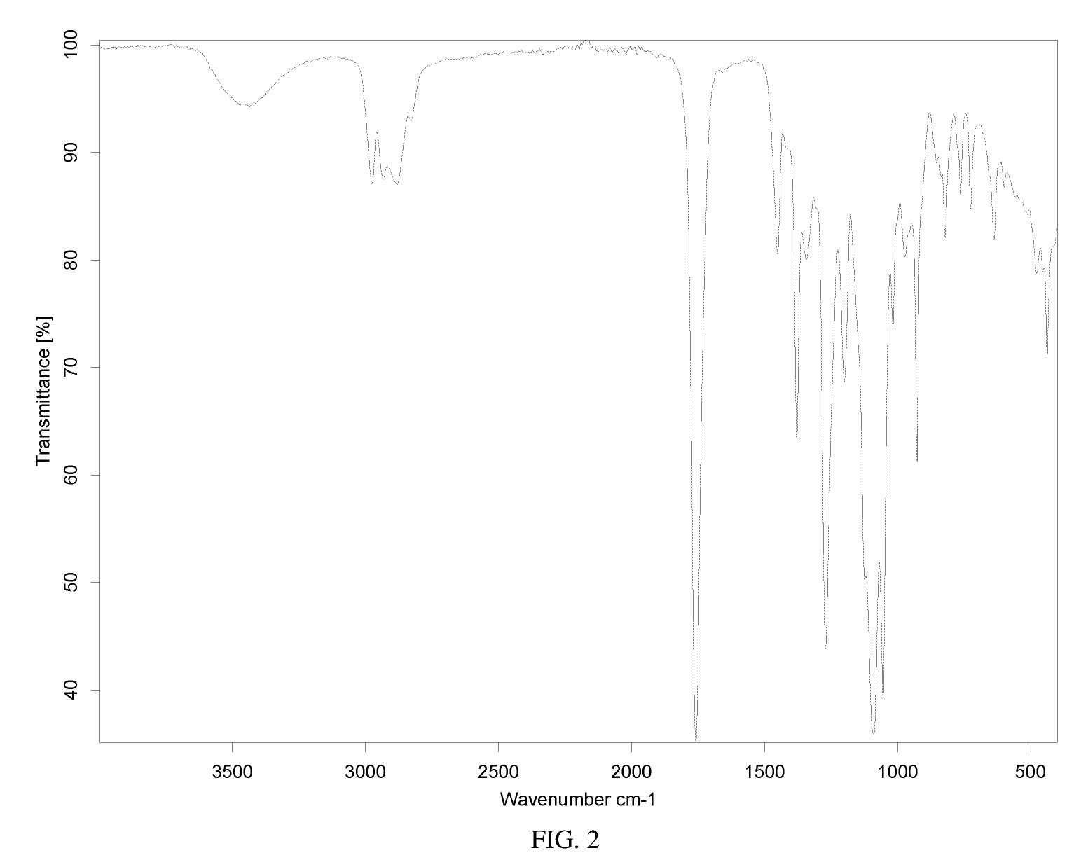

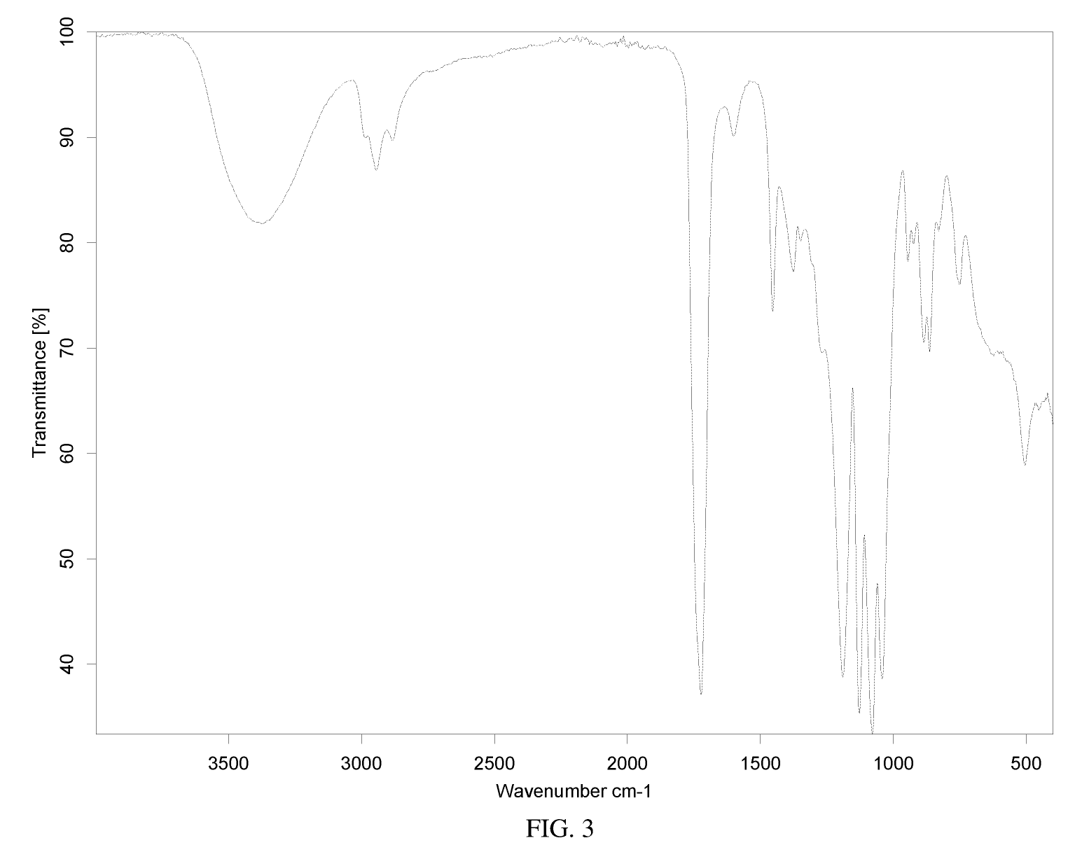

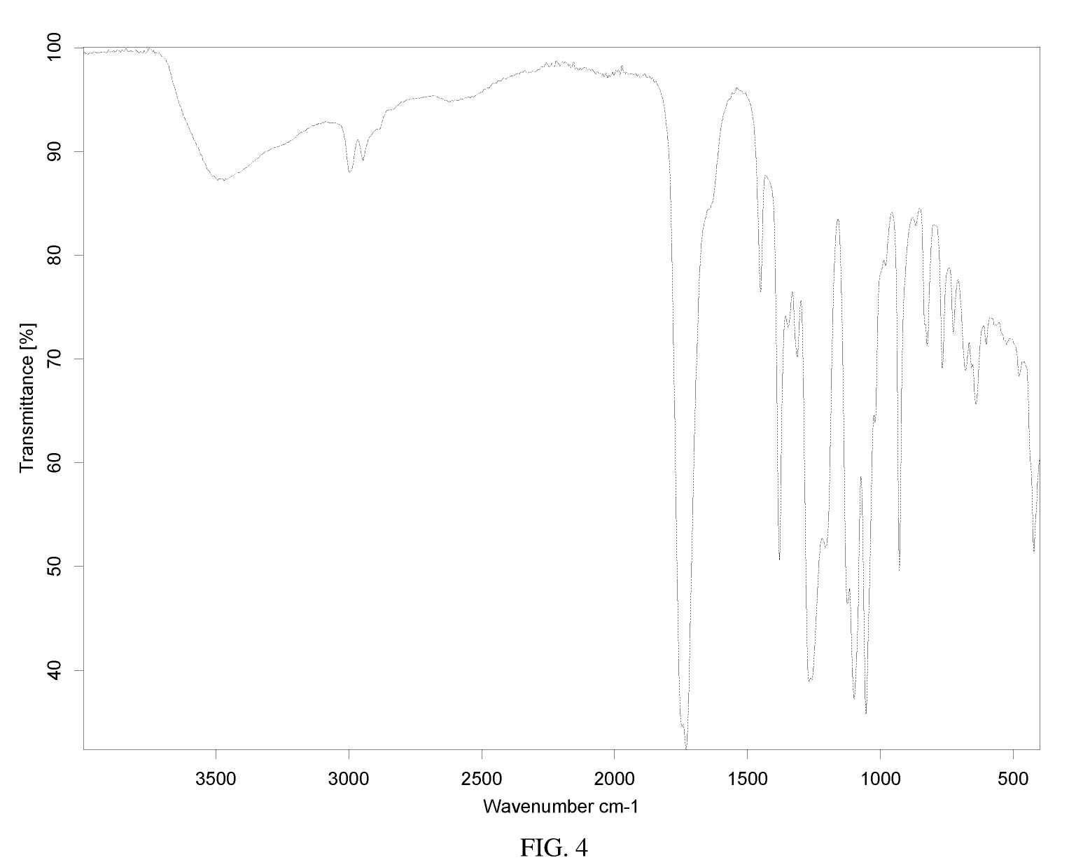

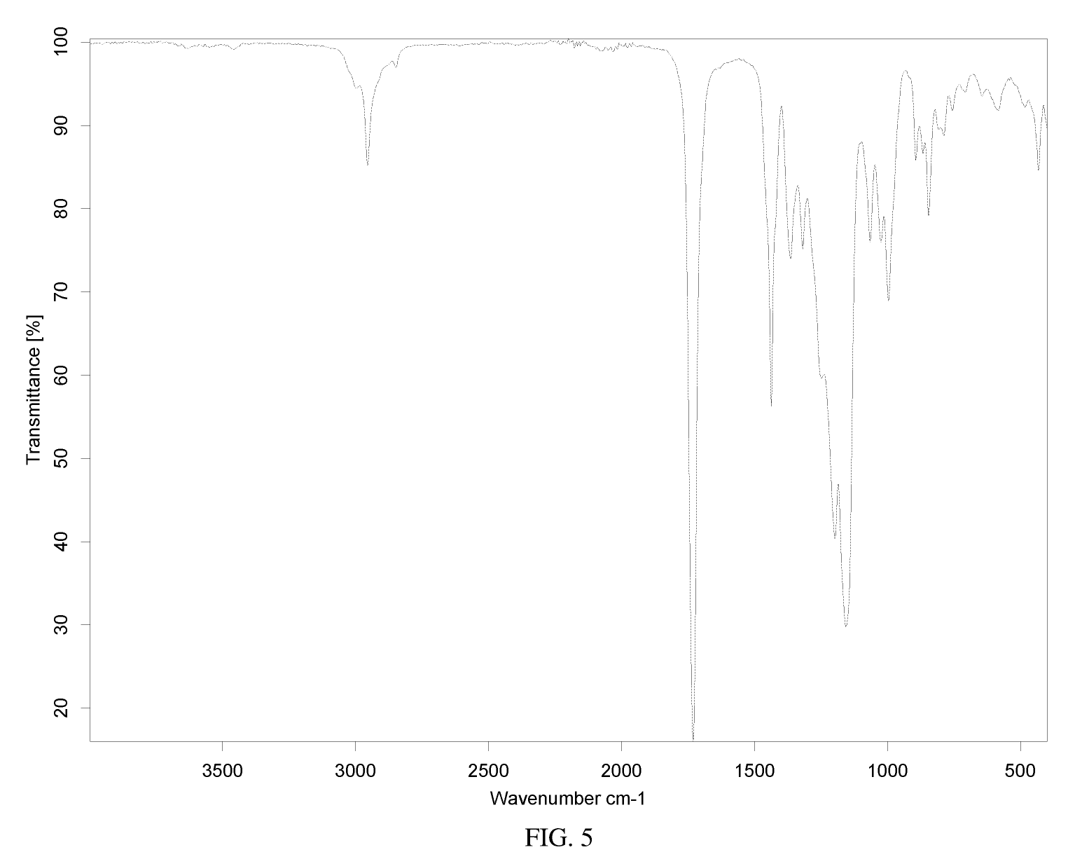

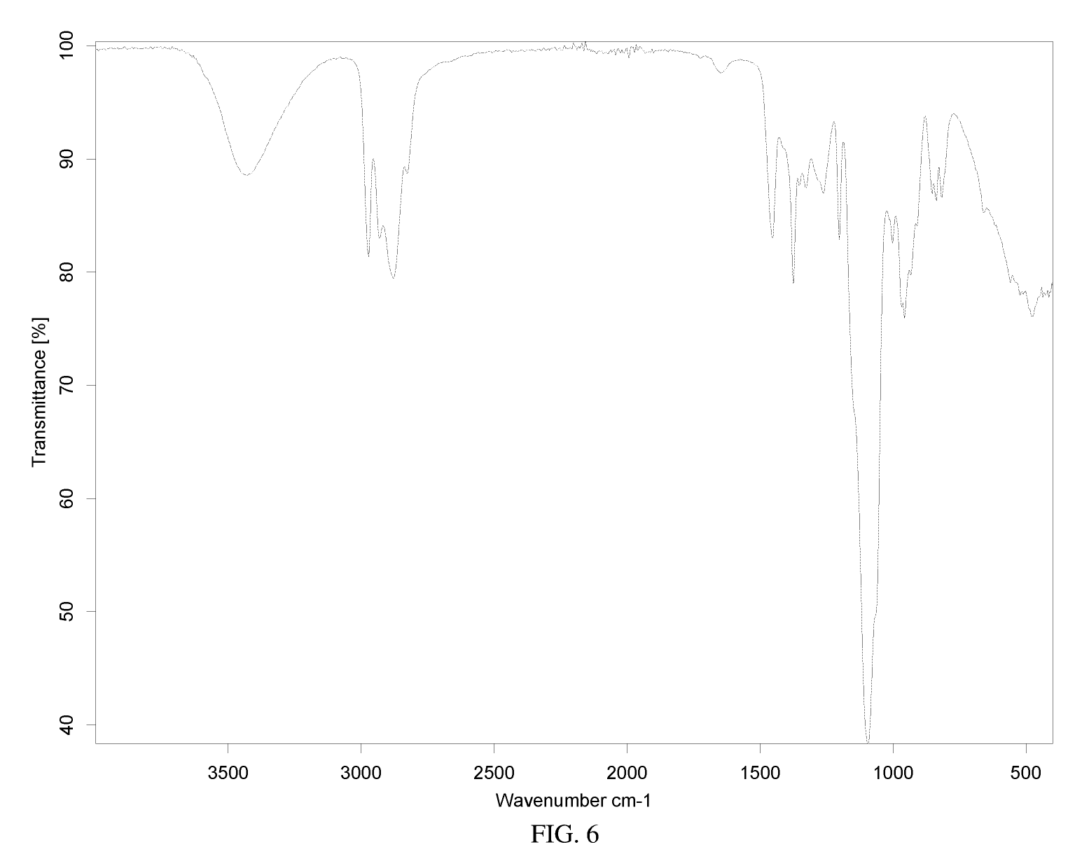

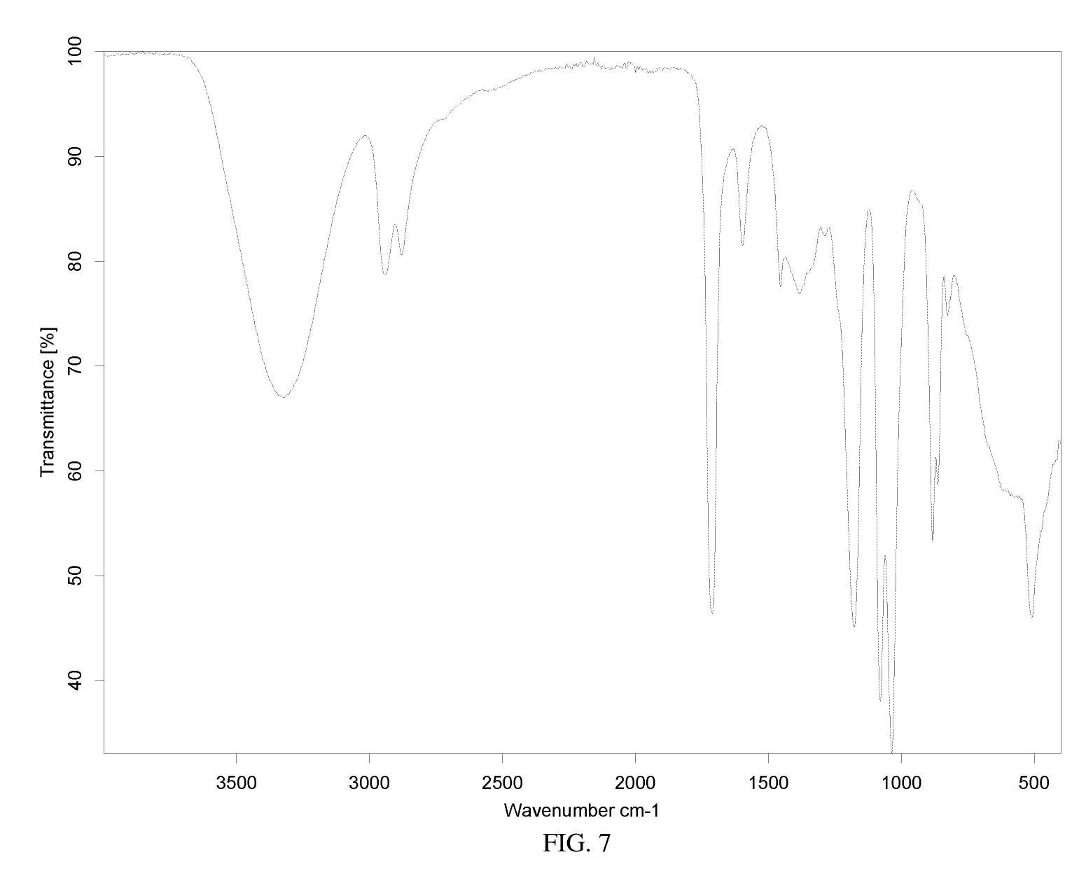

Various ACTs of the type disclosed herein were prepared and the infrared spectra of these compositions determined. Specifically the infrared spectra of an ACT comprising lactide, dimethyl glutarate, dimethyl succinate, and dimethyl adipate is shown in FIG. 1; the infrared spectra of an ACT comprising lactide and dipropylene glycol monomethyl ether is shown in FIG. 2; the infrared spectra of an ACT comprising lactide, a hydrolysable ester of formic acid and a glycol is shown in FIG. 3; the infrared spectra of an ACT comprising lactide is shown in FIG. 4; the infrared spectra of an ACT comprising dimethyl glutarate, dimethyl succinate, and dimethyl adipate is shown in FIG. 5; the infrared spectra of an ACT comprising lactide and dipropylene glycol monomethyl ether is shown in FIG. 6; and the infrared spectra of an ACT comprising lactide, a hydrolysable ester of formic acid and a glycol is shown in FIG. 7.

### Example 2

Three ACT formulations, designated Samples A, B, and C are given in Table 2.

TABLE 2

| | Sample A | Sample B | Sample C |
|---|---|---|---|
| | (wt %) | (wt %) | (wt %) |
| Lactide | 50 | 50 | 50 |
| Rhodiasolve RPDE | 50 | | |
| (from Solvay) | | | |
| Dipropylene glycol | | 50 | |
| monomethyl ether | | | |
| BIO-ADD 910 (from | | | 50 |
| Shrieve) | | | |

Lactide was commercially available from Natureworks, RHODIASOLVE RPDE is a classical dibasic ester solvent commercially available from Solvay and BIO-ADD 910 is a delayed acid source available from Shrieve Chemical Products. Twelve samples were prepared according to Table 3. These samples were place in an oven heated to between 145° F. to 155° F. for 48 hours.

TABLE 3

| Sample # | 1 | 2 | 3 | 4 | 5 | 6 | 7 | 8 | 9 | 10 | 11 | 12 |
|---|---|---|---|---|---|---|---|---|---|---|---|---|
| Fresh water | 50 ml | 50 ml | 50 ml | | | | | | | | | |
| 19.2 | | | | 50 ml | 50 ml | 50 ml | | | | | | |
| calcium | | | | | | | | | | | | |
| bromide/ | | | | | | | | | | | | |
| zinc | | | | | | | | | | | | |
| bromide | | | | | | | | | | | | |
| 14.2 | | | | | | | 50 ml | 50 ml | 50 ml | | | |
| calcium | | | | | | | | | | | | |
| bromide | | | | | | | | | | | | |
| 12.5 sodium | | | | | | | | | | 50 ml | 50 ml | 50 ml |
| bromide | | | | | | | | | | | | |
| Calcium | 1 g | 1 g | 1 g | 1 g | 1 g | 1 g | 1 g | 1 g | 1 g | 1 g | 1 g | 1 g |
| carbonate | | | | | | | | | | | | |
| Sample A | 5 ml | | | 5 ml | | | 5 ml | | | 5 ml | | |
| Sample B | | 5 ml | | | 5 ml | | | 5 ml | | | 5 ml | |
| Sample C | | | 5 ml | | | 5 ml | | | 5 ml | | | 5 ml |

After 48 hours there was complete dissolution of the calcium carbonate in all samples. This indicated that acid was generated to dissolve the calcium carbonate. As demonstrated in later examples, not all the ACT compositions were soluble in all brines. However, even without full solubility the ACT could dissolve the calcium carbonate.

### Example 3

The ability of ACT compositions of the type disclosed herein to form stable solutions was investigated. Eight sample ACT formulations were prepared according to Table 4 and evaluated for solubility according to Table 5.

TABLE 4

| | Aqueous | Aqueous | Lactide | DPM | EGMBE | RDPE | Soluble | Stable |
|---|---|---|---|---|---|---|---|---|
| Sample | Solution | (g) | (g) | (g) | (g) | (g) | at RT | @ 24 h |
| 1 | NaBr Brine | 24 | 6 | 0 | 0 | 0 | No | No |
| 2 | NaBr Brine | 24 | 0 | 6 | 0 | 0 | Yes | Yes |
| 3 | NaBr Brine | 24 | 0 | 0 | 6 | 0 | No | No |
| 4 | Tap Water | 24 | 6 | 0 | 0 | 0 | No | No |
| 5 | Tap Water | 24 | 0 | 6 | 0 | 0 | Yes | Yes |
| 6 | Tap Water | 24 | 0 | 0 | 6 | 0 | Yes | Yes |
| 7 | NaBr Brine | 24 | 0 | 0 | 0 | 6 | No | No |
| 8 | Tap Water | 24 | 0 | 0 | 0 | 6 | No | No |

TABLE 5

| Time | | | | | | |
|---|---|---|---|---|---|---|
| after | | | | | | |
| mixing | Sample | Sample | Sample | Sample | Sample | Sample |
| (h) | 1 | 4 | 2 | 5 | 3 | 6 |
| 0 | cloudy | cloudy | cloudy | clear | clear | clear |
| 1 | biphasic | biphasic | clear | clear | clear | clear |
| 2 | biphasic | biphasic | clear | clear | clear | clear |
| 4 | biphasic | biphasic | clear | clear | clear | clear |
| 24 | separated | separated | stable | stable | separated | stable |

ACT formulations, designated Samples 9-14 were prepared according to Table 6 and inspected for solubility according to Table 7.

TABLE 6

| | Aqueous | Aqueous | Lactide | DPM | Lactide/ | Soluble |
|---|---|---|---|---|---|---|
| Sample | Solution | (g) | (g) | (g) | DPM | at RT |
| 9 | NaBr Brine | 24 | 6 | 12 | 1:2 | Yes |
| 10 | Tap Water | 24 | 6 | 12 | 1:2 | Yes |
| 11 | NaBr Brine | 24 | 6 | 18 | 1:3 | Yes |
| 12 | Tap Water | 24 | 6 | 18 | 1:3 | Yes |
| 13 | NaBr Brine | 24 | 6 | 6 | 1:1 | No |
| 14 | Tap Water | 24 | 6 | 6 | 1:1 | No |

TABLE 7

| Time | | | | | | |
|---|---|---|---|---|---|---|
| after | | | | | | |
| mixing | Sample | Sample | Sample | Sample | Sample | Sample |
| (h) | 9 | 10 | 11 | 12 | 13 | 14 |
| 0 | cloudy | cloudy, | faintly | nearly | saturated | cloudy, |
| | | opaque | cloudy | clear | solids | opaque |
| 1 | clear | clear | clear | clear | ND | ND |
| 2 | clear | clear | clear | clear | ND | ND |
| 24 | clear | clear | clear | clear | ND | ND |

What is claimed is:

1. An oilwell treatment composition comprising (i) a solubilizing agent wherein the solubilizing agent comprises a saturated compound of the formula:
H—(OCₐH₂ₐ)ₓ(OC_{b}H_{2b})_{y}—OC_{c}H_{2c+1}
where a and b are each independently 1, 3, or 4; c is 1, 2 or 3; x and y each independently, are numbers ranging from 1 to 5; (ii) a solid acid precursor and (iii) an aqueous fluid wherein the mass ratio of the solubilizing agent to the aqueous solution is within the range of about 1:3 to about 1:5 and the mass ratio of the solubilizing agent to the solid acid precursor is within the range of about 3:1 to about 2:1.

2. The composition of claim 1 wherein the solubilizing agent comprises a mixture of three or more constitutional isomers wherein each constitutional isomer comprises a single oxygen-hydrogen bond and a single alkoxy group, wherein the single alkoxy group is selected from the group consisting essentially of methoxy, ethoxy and propoxy.

3. The composition of claim 2 wherein any one constitutional isomer comprises the product of a self-condensation etherification process utilizing a single type of a glycol or derivative thereof;
wherein the mixture of the three or more constitutional isomers comprises at least two distinct glycol constitutional isomers or derivatives thereof; and
wherein the at least two distinct glycol constitutional isomers or derivatives thereof do not comprise ethylene glycol or a derivative thereof.

4. The composition of claim 1 wherein the solubilizing agent comprises dipropylene glycol monomethyl ether as a mixture of three constitutional isomers with the following structures:

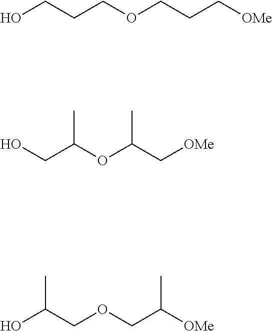

5. The composition of claim 1 wherein the aqueous fluid comprises sea water, tap water, freshwater, produced water, brine or combinations thereof.

6. The composition of claim 5 wherein the brine comprises a saturated solution of an inorganic salt consisting of monovalent cations, polyvalent cations or combinations thereof and anions consisting of chlorides, bromides, or combinations thereof.

7. The composition of claim 6 wherein the inorganic salt comprises sodium bromide.

8. The composition of claim 1 wherein the solid acid precursor is selected from the group consisting of lactide, polylactic acid, and mixtures thereof.

9. The composition of claim 1 characterized by the formation of a single visually homogenous phase within about 5 minutes of mixing.

10. The composition of claim 9 wherein the single homogeneous phase remains for at least 24 hours.

11. The composition of claim 1 further comprising dimethyl glutarate, dimethyl succinate, dimethyl adipate or combinations thereof.

12. The composition of claim 1 further comprising a surfactant.

13. A method for forming a self-degrading filter cake in a subterranean formation, comprising: placing a well drill-in and servicing fluid in a subterranean formation, the well drill-in and servicing fluid comprising a base fluid, a viscosifier, a fluid loss control additive, a bridging agent, and an in-situ filter cake degradation composition comprising a solution formed by a solubilizing agent dissolving a solid acid precursor; and forming a filter cake upon a surface within the formation. lactide, S,S-lactide, meso-lactide, glycolide, or combinations thereof.

14. A treatment composition comprising a solution made by dissolving a solid acid-precursor.

15. The composition of claim 14 further comprising a solubilizing agent wherein the solubilizing agent comprises a saturated compound of the formula:
H—(OCₐH₂ₐ)ₓ(OC_{b}H_{2b})_{y}—OC_{c}H_{2c+1}
where a and b are each independently 1, 3, or 4; c is 1, 2 or 3; and x and y each independently, are numbers ranging from 1 to 5.

16. The composition of claim 14 wherein the solid acid-precursor is selected from the group consisting of lactide, polylactic acid, and mixtures thereof.

17. The composition of claim 14 wherein the solvent used to dissolve the solid acid-precursor also generates an acid.

18. The composition of claim 14 wherein the solvent used to dissolve the solid acid-precursor is a mutual solvent, mutual solvent precursor, and mixtures thereof.

19. The composition of claim 14 wherein the solid acid precursor comprises a cyclic di-ester of an α-hydroxyacid.

20. The composition of claim 14 wherein the solvent used to dissolve the solid acid-precursor is selected from the group consisting of formic, acetic, lactic acid ester of a mutual solvent, and combinations thereof.
